(19) United States
(12) Patent Application Publication
(10) Pub. No.: US 2023/0277078 A1
(43) Pub. Date: Sep. 7, 2023

(54) ORAL PRESSURE MEASUREMENT DEVICE

(71) Applicant: Murata Manufacturing Co., Ltd., Nagaokakyo-shi (JP)

(72) Inventors: Keisuke DANNO, Nagaokakyo-shi (JP); Toru YABE, Nagaokakyo-shi (JP); Jun TAKAGI, Nagaokakyo-shi (JP); Koji TANAKA, Nagaokakyo-shi (JP)

(21) Appl. No.: 18/167,238

(22) Filed: Feb. 10, 2023

(30) Foreign Application Priority Data
Mar. 4, 2022 (JP) 2022-033688

(51) Int. Cl. A61B 5/03; A61B 5/00

(52) U.S. Cl. CPC A61B 5/038; A61B 5/7221; A61B 5/746; A61B 2562/0247; A61B 2562/043

(57) ABSTRACT

An oral pressure measurement device is provided that includes a handle and a sensor extending from the handle. The sensor includes a plate-like first pressure sensor for pressure measurement and a plate-like second pressure sensor for pressure measurement. The sensor portion further includes a first pressure dispersion plate arranged above a first principal surface of the first pressure sensor and a second pressure dispersion plate arranged above a second principal surface of the second pressure sensor. The first pressure dispersion plate and the second pressure dispersion plate are separately arranged.

### CROSS REFERENCE TO RELATED APPLICATIONS

This application claims priority to Japanese Patent Application No. 2022-033688, filed Mar. 4, 2022, the entire contents of which is hereby incorporated by reference in its entirety.

### TECHNICAL FIELD

The present invention relates to an oral pressure measurement device.

### BACKGROUND

In general, an oral pressure measurement device as described in International Publication No. 2015/115117 includes a plurality of pressure sensors, a mouthpiece, and a palate plate. Each pressure sensor detects pressure. The mouthpiece is made of an elastic material and has a U-shape. That is, the mouthpiece has a shape following an arch of an upper jaw of a measurer. The mouthpiece covers a plurality of pressure sensors. The palate plate is made of an elastic material that has a substantially quadrangular plate-like shape. Moreover, the palate plate is connectable to an inner side of the U-shape of the mouthpiece. The palate plate covers a plurality of pressure sensors different from those covered by the mouthpiece.

At the time of pressure measurement using the oral pressure measurement device described in International Publication No. 2015/115117, pressure is applied to a plurality of pressure sensors via the mouthpiece. For this reason, if pressure is applied to a particular spot in an oral cavity, pressure is applied to not only a pressure sensor facing the spot, but also one or more adjacent pressure sensors. Thus, it is difficult to measure only a pressure at a particular spot in the oral cavity with high accuracy using the oral pressure measurement device described in International Publication No. 2015/115117.

### SUMMARY OF THE INVENTION

Accordingly, an exemplary aspect of the present invention provides an oral pressure measurement device that includes a handle and a sensor portion extending from the handle. The sensor portion includes: a first pressure sensor that is configured to measure pressure and has a first principal surface; a second pressure sensor that is configured to measure pressure and has a second principal surface; a first flat plate arranged above the first principal surface of the first pressure sensor; and a second flat plate arranged above the second principal surface of the second pressure sensor. Moreover, the first flat plate and the second flat plate are separately arranged.

With the above-described configuration, a pressure acting on the first flat plate can be inhibited from being transmitted to the second flat plate. That is, a pressure when the first flat plate and the first pressure sensor are sandwiched can be inhibited from being measured by the second pressure sensor. Thus, the exemplary device is configured to detect only a pressure at a particular spot in an oral cavity with high accuracy.

As such, a pressure at a particular spot in an oral cavity can be measured by the exemplary device described herein with high accuracy.

### BRIEF DESCRIPTION OF THE DRAWINGS

FIG. 1 is a perspective view of an oral pressure measurement device according to a first exemplary embodiment;

FIG. 2 is an exploded perspective view of the oral pressure measurement device according to the first exemplary embodiment;

FIG. 3 is a transparent perspective view of a sensor cover in the oral pressure measurement device according to the first exemplary embodiment;

FIG. 4 is an exploded perspective view of an oral pressure measurement device according to a second exemplary embodiment; and

FIG. 5 is an exploded perspective view of an oral pressure measurement device according to a modification of the exemplary embodiments.

### DETAILED DESCRIPTION

### First Exemplary Embodiment

An exemplary embodiment of an oral pressure measurement device will be described below. An oral pressure measurement device to be described in the following embodiment is designed to measure pressure when the oral pressure measurement device is sandwiched between upper teeth and lower teeth, so-called occlusal force, of a user or patient. Note that the accompanying drawings may show components on an enlarged scale for ease of comprehension. Dimensional ratios between components may be different from actual ones or ones in a different drawing.

Overall Configuration of Oral Pressure Measurement Device

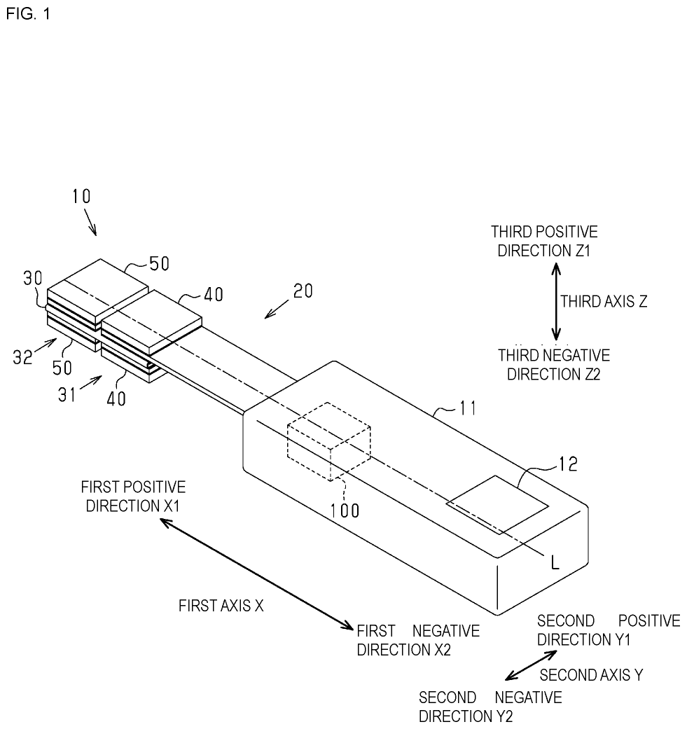

As shown in FIG. 1, an oral pressure measurement device 10 has a rod-like shape on the whole. In particular, the oral pressure measurement device 10 has a handle 11 and a sensor portion 20.

An outer shape of the handle 11 is a rod-like shape quadrangular in cross-section. That is, the handle 11 has a substantially quadrangular prism-like shape. The handle 11 is a handgrip to be gripped by a user at the time of measurement of pressure in an oral cavity.

An axis in which the handle 11 extends is assumed here as a first axis X. An axis orthogonal to the first axis X is assumed as a second axis Y. In the present embodiment, the second axis Y is an axis parallel to long sides of the handle 11 when viewed in a direction along the first axis X. An axis orthogonal to both the first axis X and the second axis Y is assumed as a third axis Z. In the present embodiment, the third axis Z is an axis parallel to short sides of the handle 11 when viewed in the direction along the first axis X. Note that one of two directions along the first axis X is assumed as a first positive direction X1 and that a direction opposite to the first positive direction X1 is assumed as a first negative direction X2. Further, one of two directions along the second axis Y is assumed as a second positive direction Y1 and a direction opposite to the second positive direction Y1 is assumed as a second negative direction Y2. Furthermore, one of two directions along the third axis Z is assumed as a third positive direction Z1 and a direction opposite to the third positive direction Z1 is assumed as a third negative direction Z2. It is also noted that a central axis L of the handle 11 is parallel to the first axis X in the present embodiment.

The handle 11 includes a display 12. The display 12 is located at a surface facing the third positive direction Z1 of outer surfaces of the handle 11. The display 12 is exposed from the handle 11 to the outside and can be viewed by an operator of the device, for example. The display 12 is designed to display various types of information including a detection value of pressure measured in the oral pressure measurement device 10. The display 12 functions as an alarm which gives notification by light.

Sensor Portion

As shown in FIG. 1, the sensor portion 20 (also referred to as a “sensor”) extends from an end face on a side facing the first positive direction X1 of the handle 11 in the first positive direction X1 on the whole. The sensor portion 20 includes a sensor cover 30, a first pressure sensor 31, a second pressure sensor 32, two first multilayer products 40, and two second multilayer products 50.

In the exemplary aspect, the sensor cover 30 has a rectangular plate-like shape long in the direction along the first axis X on the whole. An end on a side facing the first negative direction X2 of the sensor cover 30 is connected to an end on the side facing the first positive direction X1 of the handle 11. A principal surface of the sensor cover 30 faces the third positive direction Z1. That is, the principal surface of the sensor cover 30 is parallel to the central axis L of the handle 11. For purposes of this disclosure, the term “principal surface” refers to a surface with the largest area or an oppositely oriented surface of outer surfaces of a plate-like object. Moreover, the sensor cover 30 incorporates the first pressure sensor 31 and the second pressure sensor 32.

As shown in FIG. 3, the first pressure sensor 31 is located at an end portion on the side facing the first positive direction X2 in the sensor cover 30. The first pressure sensor 31 is configured to measure pressure. The first pressure sensor 31 has a substantially square (or rectangular) plate-like shape. A first principal surface 31A of the first pressure sensor 31 faces the third positive direction Z1. That is, the first principal surface 31A of the first pressure sensor 31 is parallel to the central axis L of the handle 11. When viewed in a direction along the third axis Z, two of four sides of the first pressure sensor 31 are parallel to the first axis X. When viewed in the direction along the third axis Z, the remaining two of the four sides of the first pressure sensor 31 are parallel to the second axis Y.

The first pressure sensor 31 includes one pair of plate members 36 and a resin member 37. A material for the one pair of plate members 36 is a conductive metal. Shapes of the plate members 36 are the same (e.g., symmetrically disposed with respect to one another). In the exemplary aspect, each plate member 36 has a square shape as viewed from a side facing the third positive direction Z1. Principal surfaces of the one pair of plate members 36 face each other. The principal surfaces of the plate members 36 are orthogonal to the third axis Z. The one pair of plate members 36 is connected to wiring (not shown). The one pair of plate members 36 is configured to function as electrodes of a capacitor.

A material for the resin member 37 is a synthetic resin, specifically, a dielectric elastomer. The resin member 37 is located between the principal surfaces of the one pair of plate members 36. That is, the resin member 37 fills a gap between the one pair of plate members 36. The resin member 37 has a square plate-like shape. The resin member 37 has the same square shape as the plate members 36 when viewed from the side facing the third positive direction Z1. The resin member 37 overlaps with the plate members 36 without protruding from the plate members 36. The resin member 37 is elastically deformable. That is, the resin member 37 is deformed when the resin member 37 is subject to pressure from the one pair of plate members 36. With this deformation, a distance between the one pair of plate members 36 changes. When the distance between the one pair of plate members 36 changes, a difference in potential between the one pair of plate members 36 also changes relative to the change in distance.

The second pressure sensor 32 is also configured to measure pressure. The second pressure sensor 32 has the same configuration as the first pressure sensor 31. That is, the second pressure sensor 32 includes one pair of plate members 36 and the resin member 37. The second pressure sensor 32 has a substantially square (or rectangular) plate-like shape. A second principal surface 32A of the second pressure sensor 32 faces the third positive direction Z1. That is, the second principal surface 32A of the second pressure sensor 32 is parallel to the first axis X. The second principal surface 32A of the second pressure sensor 32 faces the same direction as the first principal surface 31A of the first pressure sensor 31. That is, the second principal surface 32A of the second pressure sensor 32 is parallel to the first principal surface 31A of the first pressure sensor 31. When viewed in a direction along the third axis Z, two of four sides of the second pressure sensor 32 are parallel to the first axis X. When viewed in the direction along the third axis Z, the remaining two of the four sides of the second pressure sensor 32 are parallel to the second axis Y.

Dimensions of the second pressure sensor 32 are the same as dimensions of the first pressure sensor 31. The second pressure sensor 32 is located closer to the side facing the first positive direction X1 than the first pressure sensor 31 is. That is, the first pressure sensor 31 and the second pressure sensor 32 are lined up along the first axis X. A center of the second pressure sensor 32 and a center of the first pressure sensor 31 are located on the central axis L. The second principal surface 32A of the second pressure sensor 32 and the first principal surface 31A of the first pressure sensor 31 are located on the same plane. Note that the second pressure sensor 32 is composed of the one pair of plate members 36 and the resin member 37, like the first pressure sensor 31 as described above.

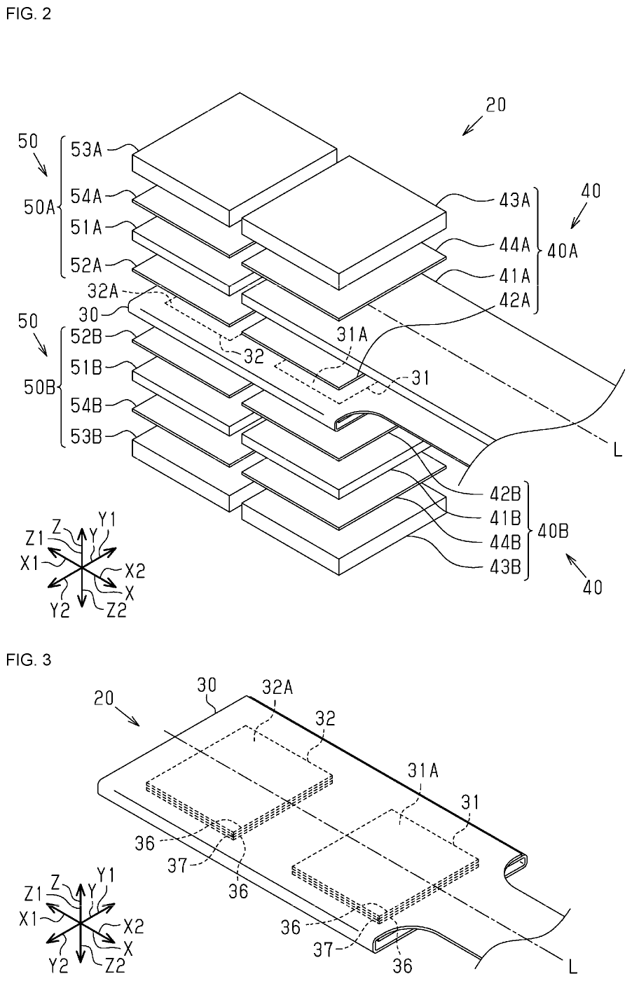

As shown in FIG. 2, a first multilayer product 40A that is one of the two first multilayer products 40 is located closer to the side facing the third positive direction Z1 than the sensor cover 30 is. The first multilayer product 40A is arranged above the first principal surface 31A of the first pressure sensor 31. For purposes of this disclosure, the expression “arranged above a principal surface” means that a multilayer product need not be in direct contact with a pressure sensor, and overlaps with the principal surface when viewed in the direction along the third axis Z (i.e., a direction normal or orthogonal in a plan view of the principal surface).

The first multilayer product 40A includes a first pressure dispersion plate 41A and a first elastic body 43A. The first pressure dispersion plate 41A has a substantially rectangular plate-like shape. A principal surface of the first pressure dispersion plate 41A faces the third positive direction Z1. When viewed in the direction along the third axis Z, two long sides of the first pressure dispersion plate 41A are parallel to the first axis X. Two short sides of the first pressure dispersion plate 41A are parallel to the second axis Y.

An end on the side facing the first negative direction X2 of the first pressure dispersion plate 41A is connected to the handle 11. A dimension in a direction along the second axis Y of the first pressure dispersion plate 41A is slightly larger than a dimension in the direction along the second axis Y of the first pressure sensor 31. The first pressure dispersion plate 41A is joined to a surface facing the third positive direction Z1 of the sensor cover 30 via an adhesive double coated tape 42A. When viewed in the third negative direction Z2, the first pressure dispersion plate 41A covers the whole of a region where the first pressure sensor 31 is present of the sensor cover 30. That is, the first pressure dispersion plate 41A is a first flat plate which is arranged above the first principal surface 31A of the first pressure sensor 31.

The first elastic body 43A has a substantially square (or rectangular) plate-like shape. A principal surface of the first elastic body 43A faces the third positive direction Z1. When viewed in the direction along the third axis Z, two of four sides of the first elastic body 43A are parallel to the first axis X. When viewed in the direction along the third axis Z, the remaining two of the four sides of the first elastic body 43A are parallel to the second axis Y. A dimension in the direction along the third axis Z of the first elastic body 43A, that is, a thickness of the first elastic body 43A is larger than a thickness of the first pressure dispersion plate 41A. A dimension in the direction along the second axis Y of the first elastic body 43A is slightly larger than the dimension in the direction along the second axis Y of the first pressure sensor 31.

The first elastic body 43A is arranged above the first pressure dispersion plate 41A. Specifically, the first elastic body 43A is joined to a surface facing the third positive direction Z1 of the first pressure dispersion plate 41A via an adhesive double coated tape 44A. The first elastic body 43A covers the whole region of the first pressure dispersion plate 41A in the direction along the second axis Y. Note that a part including the end on the side facing the first negative direction X2 in the first pressure dispersion plate 41A protrudes from the first elastic body 43A toward the side facing the first negative direction X2. A material for the first elastic body 43A is a synthetic resin. The material for the first elastic body 43A has a lower elastic modulus than a material for the first pressure dispersion plate 41A. That is, the first elastic body 43A is softer than the first pressure dispersion plate 41A.

As shown in FIG. 2, a first multilayer product 40B which is the other first multilayer product 40 is located closer to a side facing the third negative direction Z2 than the sensor cover 30 is. The first multilayer product 40B is arranged above a principal surface on the opposite side of the first pressure sensor 31 from the first principal surface 31A. The first multilayer product 40B has almost the same configuration as the first multilayer product 40A described above.

As shown, the first multilayer product 40B includes a first pressure dispersion plate 41B and a first elastic body 43B.

In an exemplary aspect, the first pressure dispersion plate 41B has a substantially square shape. A principal surface of the first pressure dispersion plate 41B faces the third negative direction Z2. When viewed in the direction along the third axis Z, two of four sides of the first pressure dispersion plate 41B are parallel to the first axis X. The remaining two of the four sides of the first pressure dispersion plate 41B are parallel to the second axis Y.

A dimension in the direction along the first axis X of the first pressure dispersion plate 41B is slightly larger than a dimension in the direction along the first axis X of the first pressure sensor 31. Further, a dimension in the direction along the second axis Y of the first pressure dispersion plate 41B is slightly larger than the dimension in the direction along the second axis Y of the first pressure sensor 31. The first pressure dispersion plate 41B is joined to a surface facing the third negative direction Z2 of the sensor cover 30 via an adhesive double coated tape 42B. When viewed in the third positive direction Z1, the first pressure dispersion plate 41B covers the whole of the region where the first pressure sensor 31 is present of the sensor cover 30.

The first elastic body 43B has a substantially square (or rectangular) plate-like shape. A principal surface of the first elastic body 43B faces the third negative direction Z2. When viewed in the direction along the third axis Z, two of four sides of the first elastic body 43B are parallel to the first axis X. When viewed in the direction along the third axis Z, the remaining two of the four sides of the first elastic body 43B are parallel to the second axis Y. A dimension in the direction along the third axis Z of the first elastic body 43B, that is, a thickness of the first elastic body 43B is larger than a thickness of the first pressure dispersion plate 41B. A dimension in the direction along the first axis X of the first elastic body 43B is slightly larger than the dimension in the direction along the first axis X of the first pressure sensor 31. Further, a dimension in the direction along the second axis Y of the first elastic body 43B is slightly larger than the dimension in the direction along the second axis Y of the first pressure sensor 31. The first elastic body 43B is joined to a surface facing the third negative direction Z2 of the first pressure dispersion plate 41B via an adhesive double coated tape 44B. When viewed in the third positive direction Z1, the first elastic body 43B covers the whole of the first pressure dispersion plate 41B. A material for the first elastic body 43B is a synthetic resin. The material for the first elastic body 43B has a lower elastic modulus than a material for the first pressure dispersion plate 41B. That is, the first elastic body 43B is softer than the first pressure dispersion plate 41B.

Moreover, a second multilayer product 50A which is one of the two second multilayer products 50 is arranged closer to the side facing the third positive direction Z1 than the sensor cover 30 is. The second multilayer product 50A is arranged above the second principal surface 32A of the second pressure sensor 32.

As further shown, the second multilayer product 50A includes a second pressure dispersion plate 51A and a second elastic body 53A.

The second pressure dispersion plate 51A has a substantially square shape. A principal surface of the second pressure dispersion plate 51A faces the third positive direction Z1. When viewed in the direction along the third axis Z, two of four sides of the second pressure dispersion plate 51A are parallel to the first axis X. The remaining two of the four sides of the second pressure dispersion plate 51A are parallel to the second axis Y.

A dimension in the direction along the first axis X of the second pressure dispersion plate 51A is slightly larger than a dimension in the direction along the first axis X of the second pressure sensor 32. A dimension in the direction along the second axis Y of the second pressure dispersion plate 51A is slightly larger than a dimension in the direction along the second axis Y of the second pressure sensor 32. The second pressure dispersion plate 51A is joined to the surface facing the third positive direction Z1 of the sensor cover 30 via an adhesive double coated tape 52A. When viewed in the third negative direction Z2, the second pressure dispersion plate 51A covers the whole of a region where the second pressure sensor 32 is present of the sensor cover 30. That is, the second pressure dispersion plate 51A is a second flat plate which is arranged above the second principal surface 32A of the second pressure sensor 32.

The second elastic body 53A has a substantially square (or rectangular) plate-like shape. A principal surface of the second elastic body 53A faces the third positive direction Z1. When viewed in the direction along the third axis Z, two of four sides of the second elastic body 53A are parallel to the first axis X. When viewed in the direction along the third axis Z, the remaining two of the four sides of the second elastic body 53A are parallel to the second axis Y. A dimension in the direction along the third axis Z of the second elastic body 53A, that is, a thickness of the second elastic body 53A is larger than a thickness of the second pressure dispersion plate 51A. A dimension in the direction along the first axis X of the second elastic body 53A is slightly larger than the dimension in the direction along the first axis X of the second pressure sensor 32. Further, a dimension in the direction along the second axis Y of the second elastic body 53A is slightly larger than the dimension in the direction along the second axis Y of the second pressure sensor 32.

The second elastic body 53A is arranged above the second pressure dispersion plate 51A. Specifically, the second elastic body 53A is joined to a surface facing the third positive direction Z1 of the second pressure dispersion plate 51A via an adhesive double coated tape 54A. When viewed in the third negative direction Z2, the second elastic body 53A covers the whole of the second pressure dispersion plate 51A. A material for the second elastic body 53A is a synthetic resin. The material for the second elastic body 53A has a lower elastic modulus than a material for the second pressure dispersion plate 51A. That is, the second elastic body 53A is softer than the second pressure dispersion plate 51A.

A second multilayer product 50B which is the other second multilayer product 50 is arranged closer to the side facing the third negative direction Z2 than the sensor cover 30 is. The second multilayer product 50B is arranged above a principal surface on the opposite side of the second pressure sensor 32 from the second principal surface 32A. The second multilayer product 50B has substantially the same configuration as the second multilayer product 50A described above. Note that, in the second multilayer product 50B, a second elastic body 53B, an adhesive double coated tape 54B, a second pressure dispersion plate 51B, and an adhesive double coated tape 52B are located in this order in the third positive direction Z1. That is, the second multilayer product 50A and the second multilayer product 50B are opposite in an order in which components are stacked.

According to the exemplary aspect, the first multilayer product 40A and the second multilayer product 50A are isolated from each other. That is, the first pressure dispersion plate 41A and the second pressure dispersion plate 51A are separately arranged. Similarly, the first elastic body 43A and the second elastic body 53A are separately arranged. Similarly, the first multilayer product 40B and the second multilayer product 50B are separately arranged.

Control Portion

As shown in FIG. 1, the oral pressure measurement device 10 includes a control portion 100 (e.g., a controller or control unit). In an exemplary aspect, the control portion 100 can be a combination of hardware and/or software configured to execute the algorithms described herein. For example, the control portion 100 can be a microcontroller, CPU or the like that is configured to execute software instructions stored on electronic memory for executing the algorithms described herein. Moreover, in the exemplary aspect, the control portion 100 is located inside the handle 11 and is connected to the first pressure sensor 31 and the second pressure sensor 32 via wiring (not shown). The control portion 100 receives a signal related to pressure to be detected by the first pressure sensor 31 as a first measurement value. The control portion 100 receives a signal related to pressure to be detected by the second pressure sensor 32 as a second measurement value.

The control portion 100 is configured to execute a first correction process of correcting a first measurement value from the first pressure sensor 31 on the basis of a first correction map. Ideally, a first measurement value from the first pressure sensor 31 should coincide with a pressure applied to act actually on the first pressure sensor 31. Meanwhile, the first measurement value may deviate slightly from the pressure applied to act actually on the first pressure sensor 31 due to shapes of the first multilayer products 40, a distance of the first pressure sensor 31 from the handle 11, and the like. The first correction process is a process for compensating for such a deviation on the basis of the first correction map.

Specifically, the control portion 100 multiplies a first measurement value from the first pressure sensor 31 by a coefficient determined in advance. The control portion 100 then treats a value obtained after the multiplication as a final first measurement value. The coefficient is determined for each segment of a range for first measurement values. A relationship between a first measurement value and the coefficient is provided as the first correction map in the form of a matrix-like map. For example, the coefficient is determined to be K1 if a first measurement value is more than or equal to X and less than X+α and is determined to be K2 if the first measurement value is more than or equal to X+α and less than X+2α. Coefficient values in the first correction map are determined in advance on the basis of tests, simulations, or the like.

The control portion 100 is also configured to execute a second correction process of correcting a second measurement value from the second pressure sensor 32 on the basis of a second correction map. Like the first correction process described above, the second correction process is a process for compensating for a deviation of a second measurement value from a pressure applied to act actually on the second pressure sensor 32 by multiplying the second measurement value by a coefficient determined in the second correction map. In the second correction map, a relationship between a second measurement value and the coefficient is provided in matrix form. Coefficient values in the second correction map are determined in advance on the basis of tests, simulations, or the like.

Note that the second pressure sensor 32 is different from the first pressure sensor 31 in, for example, a position with respect to the handle 11. For this reason, the second pressure sensor 32 is different from the first pressure sensor 31 in output characteristics of a measurement value. Thus, the coefficient values provided in the second correction map are partially or completely different from the coefficient values provided in the first correction map. As described above, since correction is performed using a different correction map with determined different values, the control portion 100 corrects a measurement value in a different manner from the first correction process, in the second correction process.

In particular, the control portion 100 is configured to estimate or determine whether a first measurement value after correction is more than or equal to or less than a threshold determined in advance and whether a second measurement value after correction is more than or equal to or less than the threshold. The threshold is determined in advance as a value smaller than a lower limit that is expected to be received as a first measurement value or a second measurement value when occlusal force is normally measured. In other words, when teeth or an oral cavity just touches the first multilayer products 40 or the second multilayer products 50, a first measurement value or a second measurement value is less than the threshold.

Assume that both the first measurement value after correction and the second measurement value after correction are more than or equal to the threshold. In this case, the control portion 100 causes the display 12 to display an error message to the effect that the first multilayer products 40 and the second multilayer products 50 are not gripped at a correct position or an icon to that effect.

Assume that both the first measurement value after correction and the second measurement value after correction are less than the threshold. In this case, the control portion 100 causes the display 12 to display an error message to the effect that the occlusal force measurement is not over or an icon to that effect.

Assume that either one of the first measurement value after correction and the second measurement value after correction is more than or equal to the threshold determined in advance and that the other of the first measurement value after correction and the second measurement value after correction is less than the threshold. In this case, the control portion 100 judges that the occlusal force measurement is correctly performed. The control portion 100 then outputs, as a result of the occlusal force measurement, only the measurement value more than or equal to the threshold of the first measurement value after correction and the second measurement value after correction to the display 12. That is, the control portion 100 causes the display 12 to display the result of the occlusal force measurement. Specifically, assume that the first measurement value after correction is more than or equal to the threshold and that the second measurement value after correction is less than the threshold. In this case, the control portion 100 outputs only the first measurement value as the measurement result to the display 12.

### Operation of First Exemplary Embodiment

At the time of measuring occlusal force with the oral pressure measurement device 10, the handle 11 is held such that the display 12 turns up. The sensor portion 20 is then inserted into an oral cavity of a user or patient. For example, in measuring occlusal force of right back teeth of a subject being measured, the oral pressure measurement device 10 is inserted such that the second pressure sensor 32 of the sensor portion 20 is located in the vicinity of the back teeth on the right side of the subject being measured.

The subject being measured interlocks his/her teeth, and applies pressure to the second multilayer products 50 in a direction of the third axis Z. The resin member 37 of the second pressure sensor 32 is then subject to the force and is deformed (e.g., compressed by the force). With the deformation of the resin member 37, electrostatic capacity between the one pair of plate members 36 changes. The oral pressure measurement device 10 detects the change in the electrostatic capacity of the second pressure sensor 32 as a change in pressure.

Then, the control portion 100 is configured to execute the second correction process to correct a second measurement value. If pressure is also detected in the first pressure sensor 31, the control portion 100 then executes the first correction process to correct a first measurement value.

Next, the control portion 100 compares the first measurement value after correction and the second measurement value after correction with the threshold. The control portion 100 executes any of the above-described processes in accordance with relationships between the first measurement value after correction and the second measurement value after correction and the threshold. For example, assume that the second measurement value after correction is more than or equal to the threshold and that the first measurement value after correction is less than the threshold. In this case, the control portion 100 outputs only the second measurement value after correction as a measurement result to the display 12.

### Effects of First Exemplary Embodiment

According to the first embodiment, pressure acting on the second multilayer products 50 can be inhibited from being transmitted to the first multilayer products 40. That is, a pressure when the second multilayer products 50 and the second pressure sensor 32 are sandwiched can be inhibited from being measured by the first pressure sensor 31. As a result, detection of only an occlusal force of an object is measured with high accuracy. Note that the same applies to a case where pressure acts on the first multilayer products 40.

According to the first embodiment, the first pressure sensor 31 is located on a side closer to the handle 11 in the direction along the first axis X. The second pressure sensor 32 is located on a side farther from the handle 11 in the direction along the first axis X than the first pressure sensor 31 is. Thus, a user of the oral pressure measurement device 10 can measure pressure with a pressure sensor which is easier to use for measurement in accordance with a positional relationship between the handle 11 and an object to be measured. Specifically, for example, the user can grip the oral pressure measurement device 10 with his/her teeth such that pressure acts on the second pressure sensor 32 at the time of measuring occlusal force of back teeth and can grip the oral pressure measurement device 10 with his/her teeth such that pressure acts on the first pressure sensor 31 at the time of measuring occlusal force of front teeth.

In the first embodiment, assume that either one of a first measurement value after correction and a second measurement value after correction is more than or equal to a threshold determined in advance and that the other of the first measurement value after correction and the second measurement value after correction is less than the threshold. In this case, the control portion 100 outputs the measurement value more than or equal to the threshold as a measurement result. According to this configuration, if a slight pressure which is not based on pressure from an object to be measured is detected by either pressure sensor, a detection result is not reflected in a measurement result. This makes it easy to precisely detect only pressure from an object to be measured.

In the first embodiment, assume that both a first measurement value after correction and a second measurement value after correction are more than or equal to a threshold determined in advance. In this case, the control portion 100 causes the display 12 to display an error message to the effect that the gripping position is not correct or an icon to that effect. According to this configuration, the control portion 100 gives notification that pressure from an object to be measured is acting on both the first pressure sensor 31 and the second pressure sensor 32 and that a correct measurement result cannot be output. Thus, a measurer (e.g. observer or user) can recognize that the measurement is being performed by an inappropriate method.

In the first embodiment, assume that both a first measurement value after correction and a second measurement value after correction are less than a threshold. In this case, the control portion 100 causes the display 12 to display a message to the effect that the occlusal force measurement is not over or an icon to that effect. According to this configuration, the control portion 100 can give notification that the subject being measured has not applied pressure. Thus, a measurer can recognize that the measurement is not complete.

Since the first pressure sensor 31 and the second pressure sensor 32 are different from each other in position, even if the first pressure sensor 31 and the second pressure sensor 32 are subject to the same pressure, a slight difference may be made between measurement values. In the first embodiment, the control portion 100 corrects a measurement value in a different manner from the first correction process, in the second correction process. As described above, since different corrections are made by the first correction process and the second correction process, correction that compensates for a slight difference in measurement value as described above is possible.

### Second Exemplary Embodiment

A second embodiment of an oral pressure measurement device will be described below. An oral pressure measurement device according to a second embodiment is different from the oral pressure measurement device according to the first embodiment in a configuration of a sensor portion. The other components are the same as in the first embodiment. Points related to a sensor portion will be described below. A description of the same components as in the first embodiment will be simplified or omitted. Note that drawings may show components on an enlarged scale for ease of comprehension. Dimensional ratios between components may be different from actual ones or ones in a different drawing.

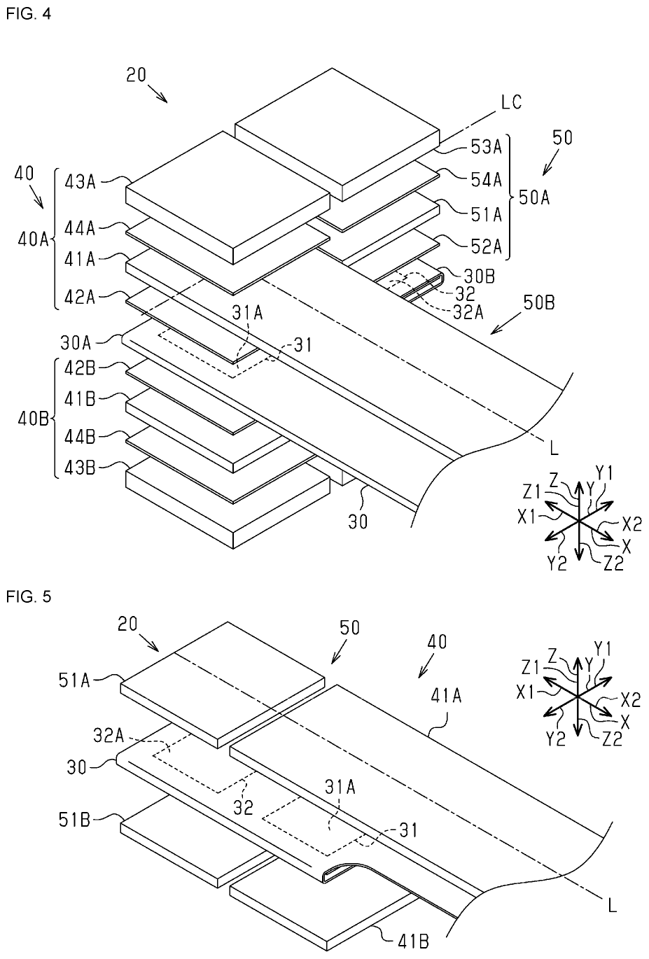

As shown in FIG. 4, a sensor portion 20 includes a sensor cover 30, a first pressure sensor 31, a second pressure sensor 32, two first multilayer products 40, and two second multilayer products 50.

The sensor cover 30 includes a first portion 30A and a second portion 30B. The first portion 30A has a rectangular plate-like shape long in a direction along a first axis X. A principal surface of the first portion 30A faces a third positive direction Z1. That is, the principal surface of the first portion 30A is parallel to the first axis X. The first portion 30A incorporates the first pressure sensor 31.

As further shown, the second portion 30B protrudes from an end on a side facing a second positive direction Y1 in the first portion 30A in the second positive direction Y1. That is, the second portion 30B extends along an orthogonal axis LC intersecting a central axis L. Note that, in the present embodiment, the orthogonal axis LC is parallel to a second axis Y orthogonal to the first axis X.

The second portion 30B has a square plate-like shape. A principal surface of the second portion 30B faces the third positive direction Z1. That is, the principal surface of the second portion 30B is parallel to the first axis X and is located on the same plane as the principal surface of the first portion 30A. The second portion 30B incorporates the second pressure sensor 32. That is, the second pressure sensor 32 is located in the second portion 30B.

The first pressure sensor 31 is located at an end portion on a side facing a first positive direction X1 in the first portion 30A. Moreover, the first pressure sensor 31 has the same configuration as in the first embodiment. That is, the first pressure sensor 31 has a substantially square plate-like shape. A first principal surface 31A of the first pressure sensor 31 faces the third positive direction Z1. That is, the first principal surface 31A of the first pressure sensor 31 is parallel to the first axis X. When viewed in a direction along the third axis Z, two of four sides of the first pressure sensor 31 are parallel to the first axis X. When viewed in the direction along the third axis Z, the remaining two of the four sides of the first pressure sensor 31 are parallel to the second axis Y.

The second pressure sensor 32 has the same configuration as the first pressure sensor 31. The second pressure sensor 32 is located closer to a side facing the second positive direction Y1 than the first pressure sensor 31 is. A center of the second pressure sensor 32 and a center of the first pressure sensor 31 are both located on the orthogonal axis LC. A second principal surface 32A of the second pressure sensor 32 and the first principal surface 31A of the first pressure sensor 31 are located on the same plane. That is, the first principal surface 31A of the first pressure sensor 31 and the second principal surface 32A of the second pressure sensor 32 are parallel to both the first axis X and the second axis Y.

A first multilayer product 40A, which is one of the two first multilayer products 40, is arranged closer to a side facing the third positive direction Z1 than the first portion 30A is. The first multilayer product 40A is arranged above the first principal surface 31A of the first pressure sensor 31. Note that the first multilayer product 40A includes an adhesive double coated tape 42A, a first pressure dispersion plate 41A, an adhesive double coated tape 44A, and a first elastic body 43A, as in the first embodiment. Arrangement of the first multilayer product 40A with respect to the first pressure sensor 31, and the like are the same as in the first embodiment. That is, the first pressure dispersion plate 41A is arranged above the first principal surface 31A of the first pressure sensor 31. The first elastic body 43A is arranged above the first pressure dispersion plate 41A.

A first multilayer product 40B, which is the other first multilayer product 40, is located closer to a side facing the third negative direction Z2 than the first portion 30A is. The first multilayer product 40B is arranged above a principal surface on the opposite side of the first pressure sensor 31 from the first principal surface 31A. Note that the first multilayer product 40B includes an adhesive double coated tape 42B, a first pressure dispersion plate 41B, an adhesive double coated tape 44B, and a first elastic body 43B, as in the first embodiment. Arrangement of the first multilayer product 40B with respect to the first pressure sensor 31, and the like are the same as in the first embodiment.

A second multilayer product 50A, which is one of the two second multilayer products 50, is located closer to the side facing the third positive direction Z1 than the second portion 30B is. The second multilayer product 50A is arranged above the second principal surface 32A of the second pressure sensor 32. Note that the second multilayer product 50A includes an adhesive double coated tape 52A, a second pressure dispersion plate 51A, an adhesive double coated tape 54A, and a second elastic body 53A, as in the first embodiment. Arrangement of the second multilayer product 50A with respect to the second pressure sensor 32, and the like are the same as in the first embodiment as described above. That is, the second pressure dispersion plate 51A is arranged above the second principal surface 32A of the second pressure sensor 32. The second elastic body 53A is arranged above the second pressure dispersion plate 51A.

A second multilayer product 50B, which is the other second multilayer product 50, is arranged closer to the side facing the third negative direction Z2 than the second portion 30B is. The second multilayer product 50B is arranged above a principal surface on the opposite side of the second pressure sensor 32 from the second principal surface 32A. Note that the second multilayer product 50B includes an adhesive double coated tape 52B, a second pressure dispersion plate 51B, an adhesive double coated tape 54B, and a second elastic body 53B, as in the first embodiment. Arrangement of the second multilayer product 50B with respect to the second pressure sensor 32, and the like are the same as in the first embodiment.

### Effects of Second Exemplary Embodiment

According to the second embodiment, two pressure sensors different in a position in a direction along the second axis Y are present. Thus, a measurer (e.g., observer or user) can measure pressure with a pressure sensor which is easier to use for measurement in accordance with, for example, by which of left and right hands a handle 11 is held, which one of occlusal force of right back teeth and occlusal force of left back teeth to measure, or the like.

According to the second embodiment, the second pressure sensor 32 located in the second portion 30B is located at a spot off the central axis L of the handle 11. Thus, pressure can be easily measured even for an object to be measured which is hard to arrange on the central axis L of the handle 11. Specifically, for example, the idea of gripping the second pressure sensor 32 located in the second portion 30B and the second multilayer products 50 with back teeth while inserting the first portion 30A of the sensor cover 30 into between the inside of a cheek and the back teeth to measure occlusal force is possible.

Modifications

The above-described exemplary embodiments can be modified in the following manners and carried out. The embodiments and the modifications below can be combined and carried out without technical contradiction as would be understood to one skilled in the art.

In particular, it is noted that the configuration of the oral pressure measurement device 10 is not limited to the examples in the embodiments described above. For example, the sensor portion 20 may have a rod-like member extending from the handle 11. In this case, the sensor cover 30 may be connected to an end portion on the opposite side of the rod-like member from the handle 11.

Moreover, the oral pressure measurement device 10 may include a bag-like cover which covers the sensor portion 20. If the oral pressure measurement device 10 has a cover, since the sensor portion 20 does not come into direct contact with an oral cavity, the sensor portion 20 can be kept in a clean state. The cover according to this modification is preferably replaceable for each measurement or is preferably removed and cleaned after each measurement.

In addition, the oral pressure measurement device 10 need not have the display 12. In this case, a measurement result from the oral pressure measurement device 10 may be output to external equipment through wired communication or wireless communication.

In an exemplary aspect, the oral pressure measurement device 10 only has an alarm that is configured to provide a notification by at least one of sound, vibration, and light. That is, the alarm is not limited to the display 12 in each of the embodiments. For example, the oral pressure measurement device 10 may include a speaker as the alarm. In this case, the speaker makes a sound and gives notification if both a first measurement value and a second measurement value are more than or equal to the threshold. The oral pressure measurement device 10 may include a vibration motor as the alarm. In this case, the vibration motor rotates and gives notification by vibration which accompanies the rotation if both a first measurement value and a second measurement value are more than or equal to the threshold.

Yet further, the oral pressure measurement device 10 is not limited to one which measures occlusal force. For example, the oral pressure measurement device 10 may be designed to measure perioral muscle pressure, such as lingual pressure, buccinator pressure, or lip pressure.

The oral pressure measurement device 10 may further include a pressure sensor in addition to the first pressure sensor 31 and the second pressure sensor 32. If a flat plate is arranged above a principal surface in the pressure sensor other than the first pressure sensor 31 and the second pressure sensor 32, multilayer products concerned with the pressure sensor only needs to be isolated from the first multilayer products 40 and the second multilayer products 50.

It should further be appreciated that the shape of the sensor portion 20 is not limited to the examples in the embodiments. The sensor portion 20 may have a portion that is curved along an arch of an upper jaw. The sensor portion 20 may have a shape in which a dimension in the direction along the second axis Y decreases in the first positive direction X1.

The first pressure sensor 31 and the second pressure sensor 32 need not be located on the same plane. For example, the sensor cover 30 may have a level difference, and the first pressure sensor 31 and the second pressure sensor 32 may be set at different positions in the direction along the third axis Z.

The first pressure sensor 31 and the second pressure sensor 32 need not be lined up on the central axis L. The first pressure sensor 31 and the second pressure sensor 32 need not be lined up on the orthogonal axis LC.

The first principal surface 31A of the first pressure sensor 31 and the second principal surface 32A of the second pressure sensor 32 need not face the same direction. That is, the first principal surface 31A of the first pressure sensor 31 and the second principal surface 32A of the second pressure sensor 32 may face different directions.

Moreover, the shapes of the first pressure sensor 31 and the second pressure sensor 32 are not limited to the examples in the embodiments. For example, the first pressure sensor 31 may have a circular shape, an elliptical shape, a rectangular shape, or the like when viewed in the direction along the third axis Z.

One of the two first multilayer products 40 may be omitted. That is, in each of the embodiments, the first multilayer product 40A located on the side facing the third positive direction Z1 of the first pressure sensor 31 or the first multilayer product 40B located on the side facing the third negative direction Z2 of the first pressure sensor 31 can be omitted. The same applies to the second multilayer products 50.

The configurations of the first multilayer products 40 are not limited to the examples in the embodiments. For example, the first elastic body 43A may be omitted in the first multilayer product 40A. For example, a different layer may be present in the first multilayer product 40A instead of or in addition to the first elastic body 43A. The same applies to configurations of the second multilayer products 50.

For example, in the example shown in FIG. 5, the first multilayer product 40A does not have the first elastic body 43A and is a single layer composed of the first pressure dispersion plate 41A. Similarly, the second multilayer product 50A does not have the second elastic body 53A and is a single layer composed of the second pressure dispersion plate 51A. Note that fixation components for each pressure dispersion plate, such as an adhesive double coated tape, is not shown in FIG. 5. The pressure dispersion plate only needs to have a flat plate-like shape, and measurement accuracy is enhanced by converting pressure in a mouth into surface pressure and transmitting the pressure to a sensor. An elastic body refers to an elastically deformable substance. Although an elastic body that has a flat plate-shape is used in each of the embodiments, the present disclosure is not limited to this configuration. Alternatively, an elastic body can be structured to have a curved surface or include a protruding portion. The elastic body may have any shape as long as an injury can be inhibited from occurring due to contact of the oral pressure measurement device 10 with the inside of a mouth of a user.

The shapes of the first multilayer products 40 and the second multilayer products 50 can be appropriately changed. For example, when viewed in the direction along the third axis Z, each first multilayer product 40 may have a polygonal shape other than a quadrangular shape, a circular shape, an elliptical shape, or a shape obtained by combing these shapes. The same applies to the second multilayer products 50.

For example, an outer surface on the side facing the third positive direction Z1 of the first elastic body 43A may have an uneven shape tailored to a shape of teeth. When viewed in the direction along the third axis Z, the shapes of the first elastic body 43A and the first pressure dispersion plate 41A may be different.

The correction method in the first correction process is not limited to the examples in the embodiments. For example, the control portion 100 may have a mathematical expression suitable for correction in a simulation or the like, and substitute a first measurement value into the mathematical expression and correct the value. The same applies to the second correction process.

Processing manners of the first correction process and the second correction process may be the same.

The control portion 100 can skip the first correction process and the second correction process.

In each of the embodiments, assume that either one of a first measurement value after correction and a second measurement value after correction is more than or equal to the threshold determined in advance and that the other of the first measurement value after correction and the second measurement value after correction is less than the threshold. In this case, the control portion 100 can be configured to output the sum of the first measurement value and the second measurement value as a measurement result to the display 12. In an exemplary aspect, the control portion 100 output the sum of the first measurement value and the second measurement value as a measurement result to the display 12 if both the first measurement value and the second measurement value are more than or equal to the threshold. If pressure is detected in both the first pressure sensor 31 and the second pressure sensor 32 as described above, a situation where occlusal force is simultaneously applied to two pressure sensors is conceivable. For this reason, pressures dispersed to the pressure sensors may be added up, and a measurement result may be output.

The control portion 100 can also be configured to control the display 12 to display both the first measurement value and the second measurement value as a measurement result. That is, the oral pressure measurement device 10 may be used for the purpose of simultaneously measuring occlusal force of an object to be measured at two spots. For example, assume that, in a configuration where the first pressure sensor 31 and the second pressure sensor 32 are lined up on the orthogonal axis LC as in the second embodiment, the first pressure sensor 31 and the second pressure sensor 32 are arranged so as to be separated by an amount corresponding to a distance between left and right teeth. The first pressure sensor 31 can measure occlusal force at left teeth. The second pressure sensor 32 can measure occlusal force at right teeth. As described above, if pressures at different spots are measured, the control portion 100 may cause the display 12 to display both a first measurement value and a second measurement value as a measurement result. Note that it is possible in the case of this modification to skip, for each of a first measurement value and a second measurement value, a process related to judgment as to whether the measurement value is more than or equal to the threshold.

In the second embodiment, the second portion 30B may be located on an axis intersecting the first axis X, along which the handle 11 extends. That is, a position of the second portion 30B is not limited to the one on the axis orthogonal to the first axis X. Similarly, the second pressure sensor 32 may be located on the axis intersecting the first axis X. In other words, the first pressure sensor 31 and the second pressure sensor 32 only need to be lined up along the axis intersecting the first axis X, along which the handle 11 extends.

In general, it is noted that the exemplary embodiments described above are intended to facilitate the understanding of the present invention, and are not intended to limit the interpretation of the present invention. The present invention may be modified and/or improved without departing from the spirit and scope thereof, and equivalents thereof are also included in the present invention. That is, exemplary embodiments obtained by those skilled in the art applying design change as appropriate on the embodiments are also included in the scope of the present invention as long as the obtained embodiments have the features of the present invention. For example, each of the elements included in each of the embodiments, and arrangement, materials, conditions, shapes, sizes, and the like thereof are not limited to those exemplified above, and may be modified as appropriate. It is to be understood that the exemplary embodiments are merely illustrative, partial substitutions or combinations of the configurations described in the different embodiments are possible to be made, and configurations obtained by such substitutions or combinations are also included in the scope of the present invention as long as they have the features of the present invention.

What is claimed:

1. An oral pressure measurement device comprising:
a handle; and
a sensor extending from the handle and including:
a first pressure sensor having a first principal surface and configured to measure pressure,
a second pressure sensor having a second principal surface and configured to measure pressure,
a first flat plate disposed above the first principal surface of the first pressure sensor, and
a second flat plate disposed above the second principal surface of the second pressure sensor and that is separately arranged from the first flat plate.

2. The oral pressure measurement device according to claim 1, wherein the first principal surface of the first pressure sensor is parallel to the second principal surface of the second pressure sensor.

3. The oral pressure measurement device according to claim 1, wherein the sensor further includes:
a first elastic body disposed above the first flat plate, and
a second elastic body disposed above the second flat plate and that is separately arranged from the first elastic body.

4. The oral pressure measurement device according to claim 1, wherein:
the handle has a rod-like outer shape that extends along a first axis, and
the first pressure sensor and the second pressure sensor are aligned along the first axis.

5. The oral pressure measurement device according to claim 4, wherein the first principal surface of the first pressure sensor and the second principal surface of the second pressure sensor are parallel to the first axis.

6. The oral pressure measurement device according to claim 1, wherein:
the handle has a rod-like outer shape that extends along a first axis, and
the first pressure sensor and the second pressure sensor are aligned along a second axis that intersects the first axis.

7. The oral pressure measurement device according to claim 6, wherein the first principal surface of the first pressure sensor and the second principal surface of the second pressure sensor are parallel to both the first axis and the second axis.

8. The oral pressure measurement device according to claim 7, wherein the sensor comprises an outer shape that includes a first portion that extends from the handle along the first axis and a second portion that extends from the first portion along the second axis.

9. The oral pressure measurement device according to claim 1, further comprising a controller configured to receive a first measurement value from the first pressure sensor and a second measurement value from the second pressure sensor.

10. The oral pressure measurement device according to claim 9,
wherein the controller is configured to compare each of the first measurement value and the second measurement value to a predetermined threshold, and
wherein, if either one of the first measurement value and the second measurement value is more than or equal to the threshold, and the other of the first measurement value and the second measurement value is less than the threshold, the controller is configured to outputs only the respective measurement value that is more than or equal to the threshold as a measurement result.

11. The oral pressure measurement device according to claim 9, further comprising an alarm configured to generate a notification as one or more of a sound, a vibration, and a light.

12. The oral pressure measurement device according to claim 11, wherein the a controller is configured to generate the alarm if both the first measurement value and the second measurement value are more than or equal to a predetermined threshold.

13. The oral pressure measurement device according to claim 9, wherein the controller is configured to execute:
a first correction process to correct the first measurement value, and
a second correction process to correct the second measurement value, with the second correction process being different from the first correction process.

14. The oral pressure measurement device according to claim 13, wherein the first correction process is configured to compensate for a deviation between the first measurement value and an actual pressure applied on the first pressure sensor.

15. The oral pressure measurement device according to claim 14, wherein the deviation is based on a shape of a multilayer product that includes a pressure dispersion plate and a first elastic body.

16. The oral pressure measurement device according to claim 14, wherein the deviation is based a distance of the first pressure sensor to the handle.

17. The oral pressure measurement device according to claim 14, wherein the first correction process compensates for the deviation based on a correction map by multiplying the first measurement value by a coefficient determined according to the correction map.

18. The oral pressure measurement device according to claim 1, wherein each of the first and second pressure sensors includes a pair of plate members and a resin member disposed therebetween.

19. The oral pressure measurement device according to claim 18, wherein each of the first and second pressure sensors is configured to measure pressure based on a change in electrostatic capacity between the respective pair of plate members.

20. The oral pressure measurement device according to claim 1, further comprising a sensor cover that incorporates the first pressure sensor and the second pressure sensor.
